(19) United States
(12) Patent Application Publication
(10) Pub. No.: US 2024/0149075 A1
(43) Pub. Date: May 9, 2024

(54) AUTOMATED BRACHYTHERAPY WITH ROBOTICS AND/OR IMAGE GUIDANCE

(71) Applicant: Cowles Ventures, LLC, Spokane, WA (US)

(72) Inventors: Emily Lin, Spokane, WA (US); Steve Boni, Spokane, WA (US); Byron Stuck, Spokane, WA (US); Kevin Kelley, Spokane, WA (US)

(73) Assignee: Cowles Ventures, LLC, Spokane, WA (US)

(21) Appl. No.: 18/532,134

(22) Filed: Dec. 7, 2023

Related U.S. Application Data
(63) Continuation of application No. PCT/US2022/032632, filed on Jun. 8, 2022.
(60) Provisional application No. 63/208,346, filed on Jun. 8, 2021.

(51) Int. Cl. A61N 5/10; A61B 34/20; A61B 34/30

(52) U.S. Cl. CPC A61N 5/1007; A61B 34/20; A61B 34/30; A61N 5/103; A61N 2005/1011; A61N 2005/1012

(57) ABSTRACT

An automated brachytherapy apparatus and method include robotics and/or image guided brachytherapy.

### RELATED APPLICATIONS

This application claims the benefit under 35 USC 120 and is a continuation of Application Serial No. PCT/US2022/32632 filed Jun. 8, 2022 that in turn claims priority under and benefit under 35 USC 119(e) of U.S. Provisional Application No. 63/208,346 filed Jun. 8, 2021 and which is incorporated herein by reference.

### FIELD

The disclosure generally to a mechanism and method for radiation oncology.

### BACKGROUND

When women are treated for breast cancer (which is the most commonly diagnosed cancer in women), they can opt for a mastectomy (complete removal of the breast tissue) or a breast conservation surgery. Due to the use of widespread screening mammograms, women are diagnosed with localized and early-stage disease so that the breast conservation surgery followed by radiation treatment may be used. The typical radiation treatment is adjuvant breast radiation. While the adjuvant breast radiation results in good survival rates, adjuvant breast radiation treatment typically takes 3.5 to 7 weeks which is too long. In addition, since the adjuvant breast radiation treatment is typically provided using external beam radiation, there is a greater risk of acute skin reactions due to the healthy tissue interaction with the radiation.

As a result, accelerated partial breast irradiation may be used which results in a quicker treatment time and less radiation-induced acute skin reactions. One technique used for the accelerated partial breast irradiation is brachytherapy. In one method, radioactive sources are permanently implanted into the breast tissue at the site of the surgery wherein the radioactive sources may be high dose or low dose.

Currently there are a handful of ways to insert radioactive sources into breast tissue. One is by a free hand method, another uses a compressive template device to temporary hold insertion catheters and the last uses a locking template system and non-fixated fiducial needle. These methods are limited in that they do not ensure the sources are placed in the desired location as prescribed by the treatment plan 100% of the time. The lack of ability to place the radioactive sources in the desired location means that the remaining tumor margin is not receiving the appropriate radiation and healthy tissue is receiving unwanted radiation.

In the high dose rate brachytherapy area, a clinician would place hollow catheters into the breast to facilitate the insertion of a temporary radioactive source per a treatment plan which are then removed once the treatment is completed. The placement of these catheters may be by either free hand directly into the breast or by free hand though compressive template systems used to stereo-tactically immobilize the breast. Both Varian Medical Systems and Nucletron offer commercially available template immobilization products.

In the low dose rate brachytherapy area, one method for permanent breast radioactive seed implantation is described in detail in “First Report of a Permanent Breast103PD Seed Implant As Adjuvant Radiation Treatment for Early-Stage Breast Cancer”, Dr. Jean-Philippe Pignol et al., International Journal of Radiation Oncology Biological Physics, Vol. 64, No. 1, pp. 176-181 (2006) which is incorporated herein by reference. This method uses a nonfixated fiducial needle, locking template and stereotactic fixation to insert lose dose rate (LDR) radioactive source strands into the treatment site. In this method, the fiducial needle can migrate/move once inserted thus changing the depth at which the source strands are deployed. In addition, the system is very cumbersome to use and is not user intuitive. Typically, 2 to 4 weeks after the procedure, the position of the radioactive source implants are confirmed via imaging guidance and the treatment planning software. Overall, the system is a manual and simple process.

It is desirable to have an automated brachytherapy system (that may include a robotic system and/or an image guided system) that makes current brachytherapy procedures more efficient by automating certain processes.

### BRIEF DESCRIPTION OF THE DRAWINGS

FIG. 1 illustrates an automated brachytherapy apparatus that has a robotic system with a patient lying supine on a table;

FIG. 2 illustrates a method of breast brachytherapy using the apparatus shown in FIG. 1;

FIG. 3 illustrates another embodiment of the automated brachytherapy apparatus that has a robotic system with a patient lying prone on a table;

FIG. 4 illustrates another method of breast brachytherapy using the apparatus shown in FIG. 3;

FIG. 5 illustrates the automated brachytherapy apparatus that has an image-guided system with a patient; and

FIG. 6 illustrates a method for performing breast brachytherapy using the apparatus shown in FIG. 5.

### DETAILED DESCRIPTION OF ONE OR MORE EMBODIMENTS

The following disclosure is particularly applicable to radioactive source implantation into breast tissue during breast brachytherapy and it is in this context that the disclosure will be described. It will be appreciated, however, that the apparatus and method has greater utility since the device can be used with other devices besides the template, needles, and radioactive sources disclosed below and may be used to implant various radioactive sources in various different tissues.

The disclosed automated brachytherapy apparatus and method makes current brachytherapy procedures more efficient by automating certain processes. In one embodiment, the automated brachytherapy apparatus may have a robotic system that will improve the reproducibility of the procedure and ensure that the sources are reliably and consistently inserted in an exact position per a patient prescription treatment plan from patient to patient as well as improve the ease-of-use of the device and procedure. In another embodiment, the automated brachytherapy apparatus may have an image guided system that will improve the reproducibility of the procedure and ensure that the sources are reliably and consistently inserted in an exact position per a patient prescription treatment plan from patient to patient as well as improve the ease-of-use of the device and procedure. In another embodiment, the automated brachytherapy apparatus may include both the robotic system and the image guided system.

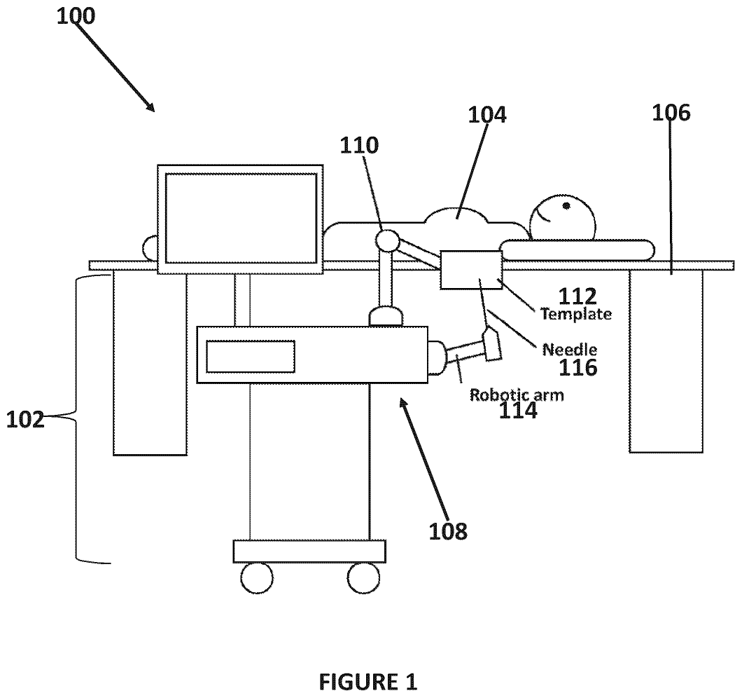

FIG. 1 illustrates an automated brachytherapy apparatus 100 that has a robotic system 102 with a patient 104 lying supine on a table 106. The robotic system 102 may have a controller 108 that is located adjacent to the table 106 and may be a computer based controller that has a processor, a display as shown, memory and a plurality of lines of code/instructions executed by the processor to implement the treatment plan (and implantation of the radioactive sources) and control the portions of the robotic system 102 as described below. The controller 108 may include an integrated treatment plan with robotic system and the treatment plan includes position of template and the robotic arm is preprogrammed to move accordingly to position the template for the particular patient. The position of template may be automatically adjusted by editing parameters in the treatment plan software or in response to patient positioning and other conditions. Machine vision guides robotic arm to move brachytherapy template to the treatment site and sensors in or near the treatment site are used to guide the movement of the robotic arm.

The plurality of lines/code in the controller 108 may control the brachytherapy procedure. The control may include program input via interface or software—e.g., the user inputs coordinates for the position of the brachytherapy device and automatic control—e.g., automatic process of inserting and steering needles to the target site.

The robotic system 102 may have a first robotic arm 110 connected to the controller 108 and also connected to a template 112 and the first robotic arm 110 programmatically adjusts the position of the template 112 adjacent to the patient 104 under control of the controller 108. The template is positioned adjacent to the patient 104 so that one or more fiducial needles and one or more radioactive source needles may be inserted into the breast tissue of the patient 104 in order to implant the radioactive sources into the breast tissue. The robotic arm 110 may move the template 112 with one or more degrees of freedom and medical imaging (that is part of the controller 102) may attach to the robotic arm and move with it.

Each fiducial needle may be inserted into the breast tissue through the template to secure the template relative to the breast tissue. Once the one or more fiducial needles are placed into the breast tissue, each radioactive source needle may contain one or more radioactive sources and the needle can be inserted into the breast tissue in order to position/place each radioactive source into the breast tissue according to the treatment plan. Examples of the information found in treatment plans include but are not limited to, number of radioactive sources and needles, implant locations of the radioactive source, the template coordinates that correspond to the implant location, the depth of insertion of the fiducial and radioactive source needles, the position of the template and fiducial needle with respect to the patient, markings on the patient, the table, or some other reference point.

The user places the fiducial needle and the radiation source needles according to the treatment plan but can adjust the locations depending on other factors such as changes in the patient anatomy. In various embodiments, each fiducial needle may be locked onto the template after the fiducial needle is positioned while each radioactive source needle is not lockable relative to the template because the template 112 has an array of holes through which the needles slide wherein there are a set of fiducial needle holes that are lockable and there is a set of radioactive source needle holes that are not lockable.

The robotic system 102 may also have a second robotic arm 114 that is connected to the controller 108 and to a needle 116 and the second robotic arm 114 programmatically inserts either/both of the fiducial needle or radioactive source needle through the template 112 into the breast tissue of the patient 104 under control of the controller 108. The needle(s) are positioned into the breast tissue (or other tissue being treated) of the patient 104 so that one or more radioactive sources are implanted into the tissue being treated. An example of the robotic arm is a mechanical arm with one or more multi-axis joints so that the arm can move in one or more degrees of freedom. One end of the arm can attach to the tools needed for the brachytherapy procedure, such as an attachment fixture for templates and needles or a mechanical hand with two or more claws that inserts the radioactive source needle and implants the radioactive sources. The arm has electronic components that connect to a system, where it takes input from the user to output movement in a given direction and position. The user can input the parameters on a controller that can take the form of a keyboard, mouse, joystick, or a touch interface on a physical or virtual device.

In some embodiments, the apparatus 100 may be used to insert the fiducial and radioactive source needles without a template in which the second robotic arm 114 inserts needles, sensors with feedback on applied forces (in the second robotic arm 114 or associated with the second robotic arm 114) guide the needle insertion force and machine vision automates the needle insertion.

In some embodiments, the controller 108 may include a mechanism to locate a template or locate needles or locate both needles and template when used in conjunction with each other. That mechanism may be integrated medical imaging technology with treatment plan with software analysis that compares the treatment plan and location of the template and needles, continuous monitoring of location with real-time medical imaging technology and automated verification via machine vision.

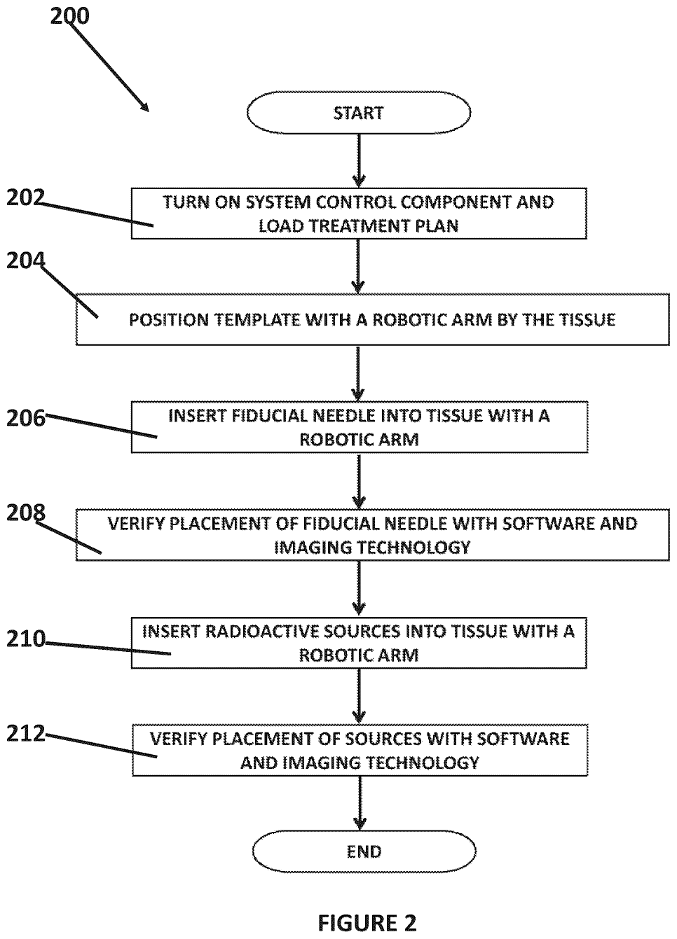

FIG. 2 illustrates a method 200 of breast brachytherapy using the apparatus shown in FIG. 1. To start the method, the controller may be turned on and a treatment plan for the particular patient being treated may be loaded into the controller (202). Based on the treatment plan and the controller, the robotic arm may position the template adjacent the tissue into which the radioactive source are being implanted (204). Once the template is positioned, a fiducial needle may be inserted into the tissue through the template using a robotic arm (206). The placement of the fiducial needle may be verified with software and imaging that are part of the controller (208). If the position is not verified, the fiducial needle may be re-positioned. Once the fiducial needle position is verified, radioactive source needle(s) may be inserted into the tissue through the template using a robotic arm (210) so that one or more radioactive sources may be placed into the tissue at the positions per the treatment plan. In one example, the arm withdraws its hold on the needle once the needle is inserted in position inside the body. The end of the needle is where the end of the radioactive source should be. The user manually retracts the needle so that the sources are released in position. In another example, the arm positions the needle in the body and then retracts the needle so that the sources are released inside the body. The placement of the radioactive sources in the tissue may be verified with software and imaging that are part of the controller (212). In this method, the positioning of the template per the treatment plan and the placement of the radioactive sources in the tissue are automated.

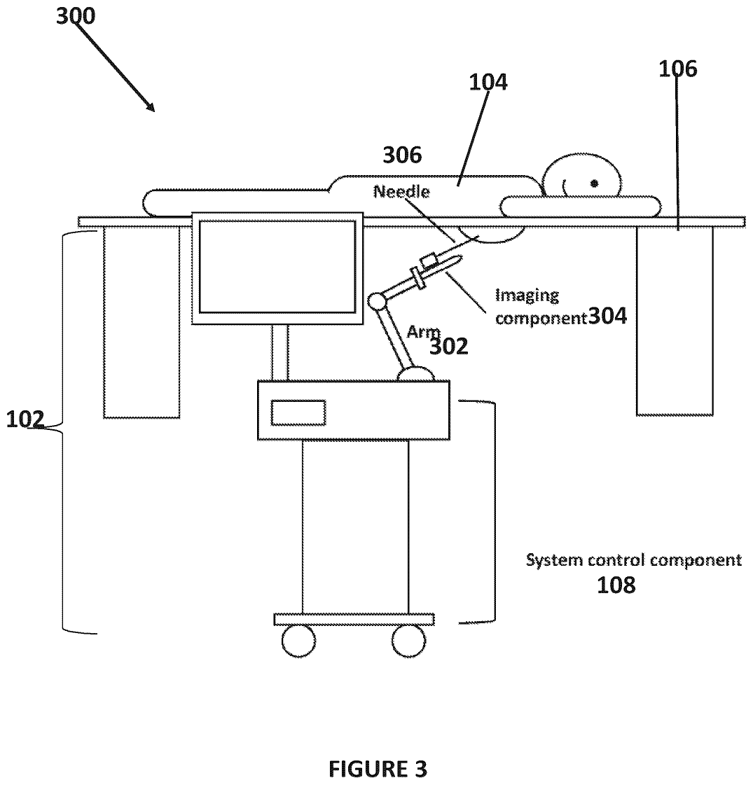

FIG. 3 illustrates another embodiment of the automated brachytherapy apparatus 300 that has a robotic system with the patient 104 lying prone on the table 106. As with the embodiment in FIG. 1, this embodiment has the robotic system 102 that has the controller/system control component 108 that has the same characteristics as the controller 108 in FIG. 1. In this embodiment, the robotic system 102 may have a robotic arm 302 connected to the controller 108 and whose opposite end has an imaging element/imager 304 and a needle connector 306 (that holds a needle that may be either a fiducial needle or a radioactive source needle). The robotic arm 302 based on the treatment plan and the imaging element may position the needle connector 306 so that radioactive sources may be implanted in the tissue of the patient. In this embodiment, the table 106 may have a void through which the tissue sits so that the radioactive sources may be implanted. The imaging element used may be ultrasound that takes the form of a needle or is integrated with the fiducial needle so that it can be inserted inside the breast as a reference point. The user operates the controller on the system control component to move the arm in position. An example of the robotic arm is a mechanical arm with one or more multi-axis joints so that the arm can move in one or more degrees of freedom. One end of the arm can attach to the tools needed for the brachytherapy procedure, such as an attachment fixture for needles and imaging element or a mechanical hand with two or more claws that inserts the radioactive source needle and implants the radioactive sources. The arm has electronic components that connect to a system, where it takes input from the user to output movement in a given direction and position. The user can input the parameters on a controller that can take the form of a keyboard, mouse, joystick, or a touch interface on a physical or virtual device.

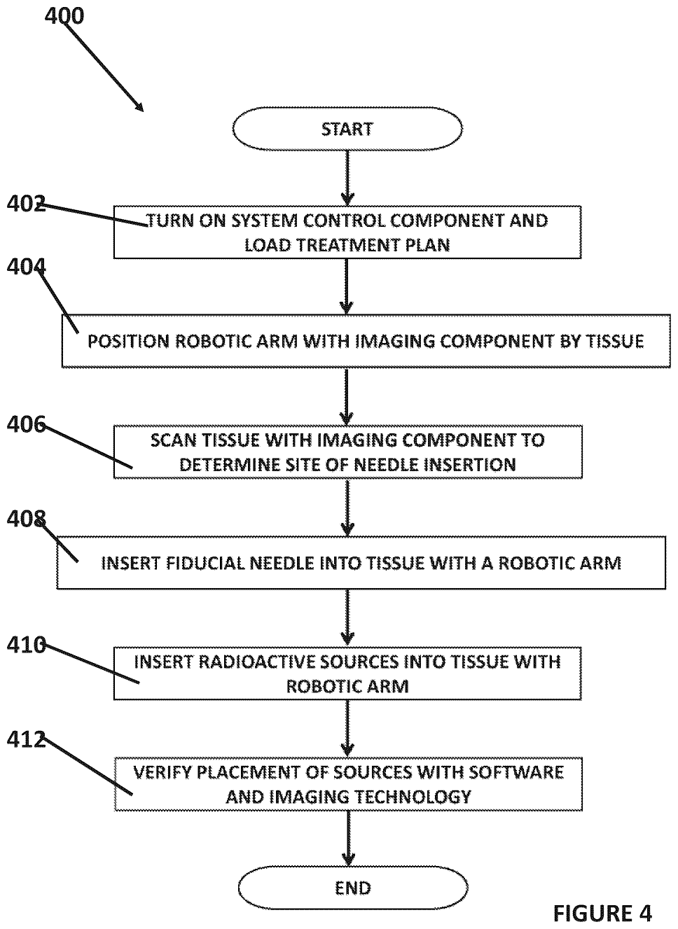

FIG. 4 illustrates another method 400 of breast brachytherapy using the apparatus shown in FIG. 3. As with the above method, the initial process is to turn on the controller and load the treatment plan (402) to the particular patient being treated. The robotic arm is then positioned near the tissue to be treated using the controller and treatment plan (404). The tissue may then be scanned using the imaging element to determine the correct site(s) of needle insertion into the tissue (406). The one or more fiducial needle(s) may then be inserted into the tissue with the robotic arm (408). The position of the fiducial needle is verified with software and imaging that are part of the system control component. The fiducial needle serves as a reference point for the placement of the radioactive source needles. Once the fiducial needle is positioned, radioactive source needle(s) may be inserted into the tissue through the template using a robotic arm (410) so that one or more radioactive sources may be placed into the tissue at the positions per the treatment plan. The placement of the radioactive sources in the tissue may be verified with software and imaging that are part of the controller (412). In this method, the positioning of the placement of the radioactive sources in the tissue are automated. The system control unit and software analyze data from imaging element, the position of the fiducial needle, and other sensors to steer the radioactive source needles into the treatment site. Using this data, the system control unit and software can also adjust the implant location from the treatment plan if the user decides that it is necessary. An example of the scan of tissue with ultrasound imaging shows the placement and insertion depth of the fiducial needle with respect to the lumpectomy cavity or treatment site, as well as the placement, insertion depth, and parallelism of the 10 radioactive source needles with respect to the fiducial needle. Permanent breast seed implant brachytherapy seeds were found to be implanted, on average, 9+/−5 mm from the planned location (Morton et al., 2016).

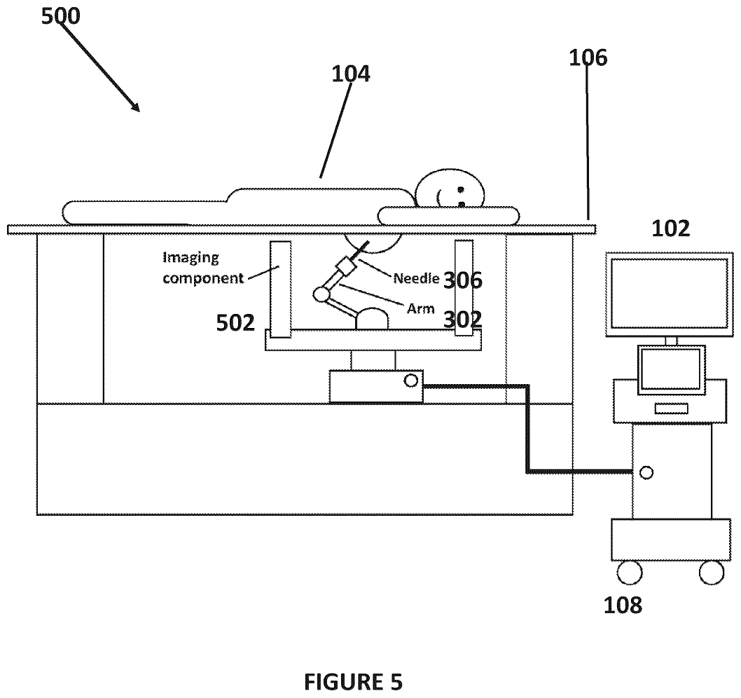

FIG. 5 illustrates the automated brachytherapy apparatus that has an image-guided system with a patient 104 lying prone on the table 106. As with the embodiment in FIG. 1, this embodiment has the robotic system 102 that has the controller/system control component 108 that has the same characteristics as the controller 108 in FIG. 1. In this embodiment, the robotic system 102 may have a robotic arm 302 connected to the controller 108 and whose opposite end has an imaging element/imager 502 and a needle connector 306 (that holds a needle that may be either a fiducial needle or a radioactive source needle). The robotic arm 302 based on the treatment plan and the imaging element may position the needle connector 306 so that radioactive sources may be implanted in the tissue of the patient. In this embodiment, the table 106 may have a void through which the tissue sits so that the radioactive sources may be implanted. The imaging element used may be CT that can revolve around the breast for different views. The user operates the controller on the system control component to move the arm in position. An example of the robotic arm is a mechanical arm with one or more multi-axis joints so that the arm can move in one or more degrees of freedom. One end of the arm can attach to the tools needed for the brachytherapy procedure, such as an attachment fixture for needles and imaging element or a mechanical hand with two or more claws that inserts the radioactive source needle and implants the radioactive sources. The arm has electronic components that connect to a system, where it takes input from the user to output movement in a given direction and position. The user can input the parameters on a controller that can take the form of a keyboard, mouse, joystick, or a touch interface on a physical or virtual device.

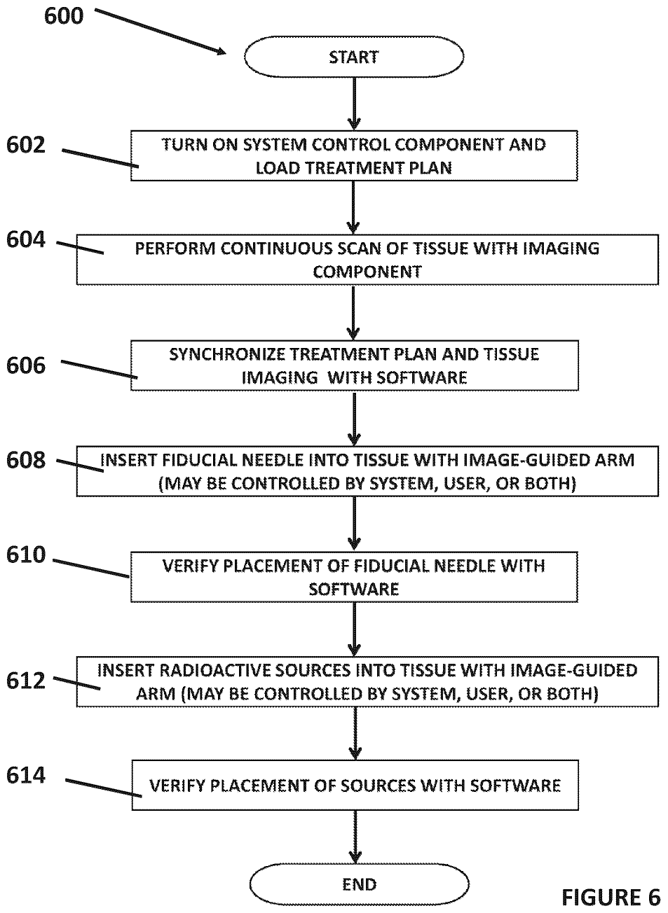

FIG. 6 illustrates a method for performing breast brachytherapy using the apparatus shown in FIG. 5. As with the above method, the initial process is to turn on the controller and load the treatment plan (602) to the particular patient being treated. The tissue may then be scanned continuously using the imaging element to determine the correct site(s) of needle insertion into the tissue (604). The imaging scans and treatment plan are synchronized with software so that the placement of the needles in the treatment site can be determined. The system control unit and software analyze data from imaging element, the position of the IO fiducial needle, and other sensors to steer the radioactive source needles into the treatment site. Using this data, the system control unit and software can also adjust the implant location from the treatment plan if the user decides that it is necessary. An example of the scan of tissue with imaging shows the placement and insertion depth of the fiducial needle with respect to the lumpectomy cavity or treatment site, as well as the placement, insertion depth, and parallelism of the radioactive source needles with respect to the fiducial needle. The one or more fiducial needle(s) may then be inserted into the tissue with the image-guided robotic arm (608). The position of the fiducial needle is verified with software and imaging that are part of the system control component. The fiducial needle serves as a reference point for the placement of the radioactive source needles. In one example, the system displays a real-time image of the treatment site with the needle. The user can visualize the location and depth of the needle and control the arm with a keyboard, buttons, and/or mouse. In another example, the user uses a controller such as a joystick to operate the arm with guidance from the system on depth and location. In another example, the user uses a controller such as a touch interface to insert the fiducial needle with guidance from the system on depth and location. Once the fiducial needle is positioned, software verifies the placement of the fiducial needle (610). In one example, the software analyzes the imaging scans for anatomical regions such as cavity size, ribs, skin and compares the placement of the fiducial needle in the scan to the placement specified in the treatment plan. Radioactive source needle(s) may be inserted into the tissue through the template using a robotic arm (612) so that one or more radioactive sources may be placed into the tissue at the positions per the treatment plan. In one example, the robotic arm uses machine vision and algorithms to insert the needles with minimal to no user input. The arm can steer the needles into the treatment site and adjust the treatment plan according to the patient's anatomy. The placement of the radioactive sources in the tissue may be verified with software and imaging that are part of the controller (614). In this method, the positioning of the placement of the radioactive sources in the tissue are automated.

The disclosed system and method automates some or all of the processes involved in guiding the delivery of radioactive sources and can be implemented using the various devices and mechanisms described below. Certain embodiments of the invention may include but are not limited to:
1. The means to move a brachytherapy device (e.g., fiducial needle, template) with guidance from medical imaging technology.
a. Integrated medical imaging technology with system—visualization of the treatment area guides the system.
1. Traditional (Ultrasound, MRI, CT) and/or novel medical imaging technology may be used.
1. Portable CT imaging.
2. Fluorescent markers for the treatment site.
3. Nanoparticles as contrast agents.
4. Ultrasound probe that are as thin or thinner than a needle.
11. Real-time visualization of the treatment area.
1. Continuous monitoring
b. One or more arms can attach to a brachytherapy device and move with one or more degrees of freedom.
1. Medical imaging technology may attach to brachytherapy device and move with arm.
11. Brachytherapy device and one or more arms may attach to medical imaging technology.
iii. Medical imaging technology may be integrated but not attached to the brachytherapy device.
c. Arm movement may be automatically adjusted by editing parameters in the treatment plan software or in response to patient positioning and other conditions.
d. Machine vision automates arm movement.
e. Sensors in or near the treatment area are used to guide the arm.
2. The means to insert needles and/or implant radioactive sources.
a. Sensors with feedback on applied forces—e.g., the needle insertion force.
b. Machine vision automates needle insertion or source implantation.
c. An arm inserts radioactive source-loaded needles.
d. A container holds radioactive sources and is attached to an arm. The radioactive sources in the container are released according to the treatment plan.
1. Container may hold the radioactive sources in a specific configuration.
11. Container may release the radioactive sources in a sequence or simultaneously.
111. Container may hold fiducial needle.
3. The means to locate the brachytherapy device (e.g., template, needles)
a. Integrated medical imaging technology with treatment plan.
1. Software analysis compares the treatment plan and location of the template and needles.
11. Continuous monitoring of location with real-time medical imaging technology.
111. Sensors on the brachytherapy device.
1v. Location marker on the brachytherapy device.
v. 3D visualization of the device at the treatment area.
b. Automated verification via machine vision.
4. The means to control the system.
a. Manual input—e.g., the user manually turns a knob.
b. Program input via interface or software—e.g., the user inputs coordinates for the position of the brachytherapy device.
c. Automatic control—e.g., the robotic arm inserts and steers the needles to the 25 target site.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. A method for automated brachytherapy, comprising:
activating a controller for brachytherapy;
loading a treatment plan for a patient into the controller, the patient;
automatically moving, under control of the controller in response to the treatment plan for the patient, a robotic arm to a position adjacent to a tissue of the patient being treated using brachytherapy, the robotic arm having an imager and a needle connector connected to a needle wherein the needle is one of a fiducial needle for fixating the brachytherapy treatment to a surgery table and a radioactive source needle containing one or more radioactive sources to be implanted into the tissue of the patient;
scanning, using the imager, the tissue of the patient to determine a needle insertion site;
inserting, using the needle connector that is moved by the robotic arm, at least one fiducial needle into the tissue at the needle insertion site; and
inserting, using the needle connector that is moved by the robotic arm, at least one radioactive source from the radioactive source needle into a position in the tissue according to the treatment plan.

2. The method of claim 1 further comprising verifying, by the imager, the position of the at least one radioactive source in the tissue.

3. The method of claim 2, wherein inserting the at least one radioactive source in the tissue further comprising inserting, by the needle connection of the robotic arm based on the treatment plan, a radioactive source needle into the tissue, moving at least one radioactive source from the radioactive source needle into the tissue and removing the radioactive source needle from the tissue.

4. The method of claim 3 further comprising lying the patient in a prone position on the table.

5. The method of claim 1 further comprising automatically positioning, using a second robotic arm connected to the controller and based on the treatment plan, a template adjacent the tissue of the patient.

6. The method of claim 5 further comprising lying the patient in a supine position on the table.

7. The method of claim 6, wherein automatically positioning the template further comprises automatically moving the template in a horizontal and vertical direction.

8. The method of claim 5 further comprising verifying, by the imager, the position of the at least one radioactive source in the tissue.

9. The method of claim 8, wherein inserting the at least one radioactive source in the tissue further comprising inserting, by the needle connection of the robotic arm based on the treatment plan, a radioactive source needle into the tissue, moving at least one radioactive source from the radioactive source needle into the tissue and removing the radioactive source needle from the tissue.

10. An apparatus, comprising:
an imaging component; one or more robotic arms, wherein one end of the arm is capable of holding and inserting brachytherapy needles; and
a system control component so that the user can operate the one or more robotic arms and an imaging component; the system control component guiding the insertion of needles and verifying the placement of needles and radioactive sources; and wherein the system control component is capable of adjusting the placement of the radioactive sources from the treatment plan based on input from the imaging component.
